(19) United States
(12) Patent Application Publication
(10) Pub. No.: US 2024/0148660 A1
(43) Pub. Date: May 9, 2024

(54) PROCESS FOR THE MANUFACTURE AND USE OF PANCREATIN MICROPELLET CORES

(71) Applicant: AbbVie Pharmaceuticals GmbH, Ludwigshafen am Rhein (DE)

(72) Inventors: George Shlieout, Sehnde (DE); Claus-Juergen Koelln, Neustadt (DE); Frithjof Sczesny, Hannover (DE); Jens Onken, Barsinghausen (DE); Guido Ruesing, Neustadt (DE)

(21) Appl. No.: 18/414,007

(22) Filed: Jan. 16, 2024

Related U.S. Application Data
(63) Continuation of application No. 17/653,575, filed on Mar. 4, 2022.
Application No. 17/653,575 is a continuation of application No. 11/464,704, filed on Aug. 15, 2006, now Pat. No. 11,266,607.
(60) Provisional application No. 60/708,526, filed on Aug. 15, 2005.
(60) Provisional application No. 60/708,692, filed on Aug. 15, 2005.

(51) Int. Cl. A61K 9/50; A61K 9/16; A61K 38/46

(52) U.S. Cl. CPC A61K 9/5042; A61K 9/1635; A61K 38/46

(57) ABSTRACT

A process for manufacturing and using pancreatin micropellet cores and pancreatin micropellets which are substantially free of synthetic oils. In one embodiment, a pharmaceutical composition is provided comprising a pancreatin micropellet with the enteric coating being designed to deliver pancreatin to the upper portion of the intestine of a mammal for release.

### CROSS-REFERENCE TO RELATED APPLICATIONS

This application claims the benefit of U.S. Provisional Application Nos. 60/708,526 and 60/708,692 which were both filed Aug. 15, 2005 and are both hereby incorporated by reference.

### FIELD OF THE INVENTION

A process for the manufacture and use of a medicament containing pancreatin is described herein. More specifically, processes for manufacturing pancreatin micropellet cores which are substantially free of synthetic oils are described. Also described herein are pancreatin micropellets which are enteric-coated pancreatin micropellet cores.

### BACKGROUND

Pancreatin microspheres are the treatment of choice for diseases or disorders caused by digestive enzyme deficiency in mammals such as humans. This is due to the fact that high-performance pancreatin microsphere products like Creon™ provide a therapeutically effective load of active enzymes while at the same time providing properly sized microspheres capable of targeting the optimal location in the digestive tract where digestive enzyme activity will be needed, in particular the upper intestine.

Recently, governmental health authorities have initiated a reassessment of the corn-patibility of certain pharmaceutical excipients which had previously been used in the for-mulation of pancreatin-containing products and have provided advice concerning specific pharmaceutical excipients (see e.g. US Code of Federal Regulations, 21 CFR § 201.302), such as mineral oil. It is recommended today that mineral oil not be provided indiscrimi-nately to either pregnant women or infants. Thus, there is a need provide patients with a pancreatin micropellet product in compliance with the current advice of the health authorities and which does not include synthetic oils such as mineral oil.

The use of synthetic oils like paraffins, e.g. liquid paraffins (mineral oils), in particular highly liquid paraffin (light mineral oil) has previously been understood to be a necessary excipient for manufacturing pancreatin micropellet products by extrusion and subse-quent spheronisation of the extrudates. One example is described in document EP 0 583 726 (U.S. Pat. No. 5,378,462), which discloses pancreatin micropellets and their preparation with polyethylene glycol 4000, paraffin and a lower alcohol, by extrusion and subse-quent spheronisation.

United States Pat. App. No. 2004/0101562 (Maio) discloses microspheres of pancreatic enzymes with high stability and a production method thereof. A solid mixture, including one or more pancreatic enzymes, one or more hydrophilic low-melting polymers and other excipients, is heated at a temperature equal or higher than the melting temperature of said hydrophilic low-melting polymer while stirring. However, Maio emphasizes that a fundamental feature of the process described therein is the total absence of any solvents, either water or other organic solvents.

In United States Pat. App. No. 2002/0061302 a method for the treatment of diabetes by administering a physiologically acceptable enzyme mixture having lipolytic, proteolytic and amylolytic activity to a patient in need thereof is described.

US patent application No. 2004/0213847 relates to delayed pharmaceutical compositions containing proton pump inhibitors.

U.S. Pat. No. 4,786,505 teaches pharmaceutical preparations for oral use.

Further pharmaceutical preparations which may comprise pancreatin and an enteric coating are e.g. known from documents DE 19907764; EP 0 021 129 (U.S. Pat. No. 4,280,971); EP 0 035 780; U.S. Pat. Nos. 5,225,202; 5,750,148; 6,224,910; U.S. Pat. App. No. 2002/0146451 or WO 02/40045.

### SUMMARY

Accordingly, one embodiment disclosed herein is a process for making and using pancreatin micropellet cores which are substantially free of synthetic oils. Another embodiment provides pancreatin micropellets substantially free of synthetic oils which are enteric-coated pancreatin micropellet cores.

Another embodiment provides a method of treating various medical conditions such as pancreatic exocrine insufficiency, pancreatitis, cystic fibrosis, diabetes type I and diabetes type II by using the pancreatin micropellet cores and/or pancreatin micropellets obtained by the processes described herein.

Another embodiment provides a pharmaceutical composition in an oral dosage form containing a pharmacologically effective amount of pancreatin wherein the pancreatin is in the form of pancreatin micropellet cores and/or pancreatin micropellets manufactured according to the processes described herein.

Other objects, features and advantages will be set forth in the detailed description of the embodiments that follows, and in part will be apparent from the description or may be learned by practice of the claimed invention. These objects and advantages will be real-ized and attained by the processes and compositions particularly pointed out in the written description and claims hereof.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. The headings used through-out this disclosure are provided for convenience only and are not to be construed to limit the invention in any way. Embodiments illustrated under any heading may be combined with embodiments illustrated under any other heading.

Pancreatin is a mixture of different physiologically active endogenous ingredients which are derived from mammalian pancreas glands and comprised of several different digestive enzymes such as lipases, amylases and proteases. Mammalian pancreatic lipase is a valuable digestive enzyme supplement for the treatment of various medical conditions such as pancreatic exocrine insufficiency. However, pancreatic proteases and amylases also contribute to the therapeutic value of pancreatin. Pancreatin for pharmaceutical use is typically of bovine or porcine origin with porcine pancreatin being preferred.

It has now been surprisingly found that pancreatin micropellet cores which are suitable for enteric coating, are high in enzymatic activity and are substantially free of synthetic oils like paraffins, e.g. highly liquid paraffin, can be produced by the processes described herein. It has further been found that the manufacturing process described herein is an improvement when compared to known processes which use mineral oil or known processes which would e.g. need more process steps to produce pancreatin micropellet cores.

In particular, pancreatin micropellet cores can be produced by the process described herein which comprise about 10% to about 95% by weight of pancreatin, about 5% to about 90% by weight of at least one pharmaceutically acceptable binding agent and 0% to about 10% by weight of at least one pharmaceutically acceptable excipient. More specifically, pancreatin micropellet cores can be produced by the process described herein which comprise about 70% to about 90% by weight of pancreatin, about 10% to about 30% by weight of at least one pharmaceutically acceptable binding agent and 0% to about 5% by weight of at least one pharmaceutically acceptable excipient. In one embodiment, pancreatin micropellet cores can be produced which comprise about 70% to about 90% by weight pancreatin, and about 10% to about 30% by weight of at least one pharmaceutically acceptable binding agent.

For the purposes of the present disclosure, the prefix “micro” used to describe a micropellet or a microsphere means that the diameter or each of the individual dimensions (length, height, width) is equal to or less than about 5 mm. Producing pancreatin micropellet cores which are approximately spherical and have a diameter of 0.5 to 2.0 mm is preferred.

The term “synthetic oils” means unsaponifiable hydrocarbons or mixtures of hydrocarbons and comprises e.g. liquid and solid paraffins, in particular liquid paraffins (mineral oils), more particularly highly liquid paraffin (light mineral oil).

The phrase “substantially free of synthetic oils” means that the manufacturing processes described herein and used to make the pancreatin micropellet cores and/or pancreatin micropellets do not utilize one or more synthetic oils as an excipient although synthetic oils may be present as pharmaceutically acceptable trace contaminants in the pancreatin, binding agent(s), enteric coating constituents, the enzyme-friendly organic solvents and/or excipients which are used to manufacture the pancreatin micropellet cores and/or pancreatin micropellets described herein.

One embodiment described herein is a process for the manufacture of pancreatin micropellet cores, comprising the steps of:
a. preparing an extrudable mixture comprising:
   i. about 10% to about 95% pancreatin;
   ii. about 5% to about 90% of at least one pharmaceutically acceptable binding agent;
   iii. 0% to about 10% of at least one pharmaceutically acceptable excipient; and
   iv. one or more enzyme-friendly organic solvents in an amount sufficient to form an extrudable mixture;
   wherein the percentages of components are weight to weight of the pancreatin micropellet cores;
b. creating pancreatin micropellet cores from the extrudable mixture;
c. forming the pancreatin micropellet cores into approximately spherical or approximately ellipsoidal shape in the presence of additional enzyme-friendly organic solvent; and
d. removing the one or more enzyme-friendly organic solvents from the pancreatin micropellet cores such that the pancreatin micropellet cores are substantially free of the one or more enzyme-friendly organic solvents;
wherein the pancreatin micropellet cores are substantially free of synthetic oils.

Examples of pharmaceutically acceptable binding agents include polyethylene glycol 1500, polyethylene glycol 2000, polyethylene glycol 3000, polyethylene glycol 4000, polyethylene glycol 6000, polyethylene glycol 8000, polyethylene glycol 10000, hydroxypropyl methylcellulose, polyoxyethylen, copolymers of polyoxyethylen-polyoxypropylen and mixtures of said organic polymers. The foregoing list of pharmaceutically acceptable binding agents is not meant to be exhaustive, but merely illustrative as a person or ordinary skill in the art would understand that many other pharmaceutically acceptable binding agents or combination of binding agents could also be used. Polyethylene glycol 4000 is the preferred pharmaceutically acceptable binding agent. For the purposes of the present disclosure, synthetic oils are not to be regarded as suitable pharmaceutically acceptable binding agents.

Examples of suitable pharmaceutically acceptable excipients include gliding agents like magnesium stearate or calcium stearate, stearic acid, talcum and/or starch; fillers like calcium phosphate, corn starch, dextrans, dextrin, hydrated silicon dioxide, microcrystalline cellulose, kaolin, lactose, mannitol, polyvinyl pyrrolidone, precipitated calcium carbonate, sorbitol and/or talcum; disintegrating agents like Aerosil™ (silicic acid), alginic acid, amylose, calcium alginate, calcium carbonate, formaldehyde gelatin, pectic carbonate, sago starch, sodium bicarbonate and/or starch; and/or moisturizers like glycerol and/or starch. The foregoing list of pharmaceutically acceptable excipients is not meant to be exhaustive, but merely illustrative as a person or ordinary skill in the art would understand that many other pharmaceutically acceptable excipients or combination of excipients could also be used. For the purposes of the present disclosure, synthetic oils are not to be regarded as suitable pharmaceutically acceptable excipients. In one embodiment, the pancreatin micropellet cores contain no pharmaceutically acceptable excipients but can optionally contain a higher load or dose of pancreatin.

Process variations wherein the pharmaceutically acceptable excipients are present in an amount of 0% are preferred.

Enzyme-friendly organic solvents facilitate mixing and other processing procedures and may afterwards be removed, for example, by drying. Typically, after removal of the enzyme-friendly organic solvents, a certain amount of solvent remains in the pancreatin micropellet cores. The remaining solvent in the micropellet cores can comprise enzyme-friendly organic solvents, water, or a mixture of enzyme-friendly organic solvents with water. If water is present as a solvent, this will typically have been present in the pancreatin which was used as the starting material. The amount of solvent present in the pancreatin micropellet cores after removal of the enzyme-friendly organic solvents is typically less than about 5% and normally less than about 3% by weight of the pancreatin micropellet core.

Examples of suitable enzyme-friendly organic solvents are acetone, chloroform, dichloromethane or straight-chained or branched C1_ 4-alcohols, particularly methanol, ethanol, 1-propanol, 2-propanol, 2-butanol, tert-butanol or mixtures of said solvents. 2-propanol is the preferred enzyme-friendly organic solvent. For the purposes of the present disclosure, synthetic oils are not to be regarded as suitable enzyme-friendly organic solvents. The enzyme-friendly organic solvent is typically used in an amount of about 15% to about 35% by weight, preferably of about 20% to about 30% by weight, relative to the amount of pancreatin used. The foregoing list of suitable enzyme-friendly organic solvents is not meant to be exhaustive, but merely illustrative as a person or ordinary skill in the art would understand that many other enzyme-friendly organic solvents or combination of solvents could also be used.

The amounts of pancreatin, pharmaceutically acceptable binding agent(s), pharmaceutically acceptable excipient(s) and/or enzyme-friendly organic solvent may be varied by those skilled in the art to arrive at the pancreatin micropellet cores having the preferred composition and characteristics as indicated herein.

The term “substantially free of enzyme-friendly organic solvents” means that the quantity of enzyme-friendly organic solvents present in the pancreatin micropellet cores would be less than about 5% by weight of the pancreatin micropellet core.

Removal of the one or more enzyme-friendly organic solvents from the pancreatin micropellet cores in process step d.) means that said pancreatin micropellet cores are subject to conditions whereby the micropellet cores become substantially free from en-zyme-friendly organic solvents. Removal of the enzyme-friendly organic solvents can be by any method known to those of ordinary skill in the art. The preferred method is by drying. Additionally, removal of the one or more enzyme-friendly organic solvents would also typically result in the pancreatin micropellet cores containing an amount of water which is less than about 5% and typically less than about 3% by weight of the pancreatin micropellet core.

In one embodiment, the pancreatin micropellet cores are created in process step b.) by extrusion. Remarkably, an extrudable mixture is obtained even though the mixture is substantially free of synthetic oils. In process step b.), if the creating of the micropellet cores from the extrudable mixture is accomplished by means of extrusion, then the temperature preferably does not exceed about 70° C. during extrusion, more preferably the temperature does not exceed about 50° C. Also, in the event of extrusion, piercing dies are preferably used which have a hole diameter of about 0.5 mm to about 2.0 mm, preferably of about 0.7 mm to about 1.5 mm, e.g. 0.8 mm. If the extrudable mixture is extruded, then the extrudate fragments are brought to a suitable length for the forming step. This can be done e.g. by means of a cutting device arranged downstream to the extruding press in a manner known to the a person of ordinary skill in the art. The forming in process step c.) can be carried out e.g. in a customary rounding apparatus. In the rounding apparatus, the extrudate fragments are then formed into an approximately spherical or approximately ellipsoidal shape in the presence of additional enzyme-friendly organic solvent which may be the same or different than the enzyme-friendly organic solvent used in process step a).

When prepared as described herein (substantially free of synthetic oils), processing of the extrudate fragments in the rounding apparatus is improved relative to other known processes. For example, a lower amount of enzyme-friendly organic solvent needs to be added when forming the pancreatin micropellet cores into an approximately spherical or approximately ellipsoidal shape and fewer of the extrudate fragments stick to parts of the rounding apparatus when the process is practiced with an extruder and rounding apparatus.

A further embodiment comprises pancreatin micropellets which are enteric-coated pancreatin micropellet cores. For enteric coating, any enteric coating can be used which is suitable for delivery of the pancreatin micropellet cores to the upper intestine and compatible with the pancreatin micropellet cores. Examples are enteric coatings known from U.S. Pat. No. 5,378,462 or commercially available enteric coatings like Eudragit™ polymers. Preferred enteric coatings are ones that would not require the presence of synthetic oils.

It has been found that the pancreatin micropellet cores and the pancreatin micropellets produced according to the processes disclosed herein and not using synthetic oils unexpectedly show essentially the same properties as pancreatin micropellet cores and pancreatin micropellets produced according to known processes using mineral oil such as the processes disclosed in U.S. Pat. No. 5,378,462. In particular, the pancreatin micropellet cores and the pancreatin micropellets produced without using synthetic oils have a similar particle size distribution, bulk density and are obtained in similar yields as the pancreatin micropellet cores and pancreatin micropellets produced according to processes which use synthetic oils. Further, the pancreatin micropellet cores produced without using synthetic oils, when compared to similar pancreatin micropellets using synthetic oils, show similar appearances in their surface structures and a similar performance when coated with an enteric coating to give pancreatin micropellets.

In another embodiment, the enteric coating on the pancreatin micropellet cores comprises:
i) at least one film-forming agent;
ii) at least one plasticizer; and
iii) optionally at least one anti-sticking agent.

In one embodiment the enteric coating comprises between about 20% and about 30% by weight, more preferably between about 22% and about 26% by weight, yet more preferably between about 22.5% and about 25% by weight of the total composition of the pancreatin micropellet.

Film-forming agent(s), plasticizer(s) and anti-sticking agent(s) (when present) as used for preparing the enteric coating are hereinafter commonly referred to as “non-solvent coating constituents”.

Suitable film-forming agents include agar, Carbopol™ (carbomer) polymers (i.e. high molecular weight, crosslinked, acrylic acid-based polymers), carboxymethyl cellulose, carboxymethylethyl cellulose, carrageen, cellulose acetate phthalate, cellulose acetate succinate, cellulose acetate trimelliate, chitin, corn protein extract, ethyl cellulose, gum arabic, hydroxypropyl cellulose, hydroxypropylmethyl acetate succinate, hydroxypropyl methylcellulose acetate succinate, hydroxypropyl methylcellulose phthalate, methacrylic acid-ethyl methacrylate-copolymer, methyl cellulose, pectin, polyvinyl acetate phthalate, polivinyl alcohol, shellac, sodium alginate, starch acetate phthalate and/or sty-rene/maleic acid copolymer or mixtures of said film-forming polymers. Cellulose acetate phthalate, hydroxypropyl methylcellulose acetate succinate and/or methacrylic acid-ethyl methacrylate-copolymer are the preferred film-forming agents. Most preferred is hydroxypropyl methylcellulose phthalate, e.g. HP 55 or HPMCP HP-50. Synthetic oils are not to be regarded as preferred film-forming agents. The foregoing list of film-forming agents is not meant to be exhaustive but merely illustrative, as a person or ordinary skill in the art would understand that many other film-forming agents or combination of film-forming agents could also be used.

The plasticizer(s) may generally be present in an amount greater than about 1.5%, and typically in an amount of about 2% to about 20% by weight, relative to the film-forming agent. The plasticizer may contain saturated linear monohydric alcohols having 12 to 30 carbon atoms. More specifically, acceptable plasticizers include lauryl alcohol, tridecyl alcohol, myristyl alcohol, pentadecyl alcohol, cetyl alcohol, heptadecyl alcohol, stearyl alcohol, nonadecyl alcohol, arachic alcohol, behenyl alcohol, carnaubyl alcohol, ceryl alcohol, corianyl alcohol, melissyl alcohol, acetyl tributyl citrate, dibutyl sebacate, fatty acid esters of glycerol, glycerol, polyethylene glycol, propyleneglycol, sorbitan fatty acids, triacetin, triethyl citrate and mixtures of said plasticizers. Preferred plasticizers are cetyl alcohol, stearyl alcohol, triethyl citrate and mixtures thereof. When cetyl alcohol is used as a single plasticizer, it may be present in an amount of greater than about 1.5%, typically in an amount of about 2% to about 15%, preferably about 2% to about 10%, by weight relative to the film-forming agent. When triethyl citrate is used as a single plasticizer, it may be present in an amount of about 5% to about 20%, preferably about 12% to about 15%, by weight relative to the film-forming agent. Synthetic oils are not to be regarded as preferred plasticizers. The foregoing list of plasticizers is not meant to be exhaustive but merely illustrative, as a person or ordinary skill in the art would understand that many other plasticizers or combination of plasticizers could also be used.

In one embodiment the plasticizer is comprised of cetyl alcohol and triethyl citrate which are collectively present in an amount of greater than about 3%, typically in an amount of about 4% to about 20%, in particular between about 6% and about 15%, more particularly between about 7% and about 10%, by weight in relation to the film-forming agent. When the plasticizer is comprised of both cetyl alcohol and triethyl citrate, the weight to weight ratio of cetyl alcohol to triethyl citrate may be from about 0.05:1 to about 1:1, for example 0.1:1, 0.2:1, 0.3:1, 0.4:1, 0.5:1, 0.6:1, 0,7:1, 0.8:1 or 0.9:1. In particular, the ratio of cetyl alcohol to triethyl citrate in said mixture of cetyl alcohol and triethyl citrate may be from about 0.25:1 to about 0.5:1, preferably from about 0.3:1 to about 0.45:1, more preferably from about 0.35:1 to about 0.4:1, and even more preferably from about 0.38:1 to about 0.4:1 (w/w).

The enteric coating optionally comprises an anti-sticking agent. Suitable anti-sticking agents include dimethicone and castor oil. Dimethicone, in particular dimethicone 1000, is the preferred anti-sticking agent. The amount of anti-sticking agent (if present) in the enteric coating is between about 1.5% and about 3% by weight relative to the film-forming agent. Synthetic oils are not to be regarded as preferred anti-sticking agents. The foregoing list of anti-sticking agents is not meant to be exhaustive but merely illustrative, as a person or ordinary skill in the art would understand that many other anti-sticking agents or combination of anti-sticking agents could also be used.

Additional embodiments are also located in U.S. Pat. App. No.______filed on Aug. 15, 2006 and claiming the benefit of U.S. Provisional Application Nos. 60/708,526 and 60/708,692 which were both filed on Aug. 15, 2005. U.S. Pat. App. No.______is hereby incorporated by reference.

Another embodiment provides a process for the manufacture of pancreatin micropellets, comprising the steps of:
aa. providing pancreatin micropellet cores wherein the pancreatin micropellet cores are substantially free of synthetic oils;
bb. providing an enteric-coating solution comprising
   i. one or more film-forming agents;
   ii. a plasticizer in an amount greater than about 1.5% by weight relative to the one or more film-forming agents; and
   iii. optionally, at least one anti-sticking agent, and
   iv. one or more enzyme-friendly organic solvent(s);
cc. coating the pancreatin micropellet cores with the enteric-coating solution wherein the temperature of the pancreatin micropellet cores during coating is kept at a temperature suitable for applying the enteric-coating solution; and
dd. drying the coated pancreatin micropellet cores.

In the foregoing process for producing pancreatin micropellets, the film-forming agent(s), the plasticizer(s), the anti-sticking agent(s) and the enzyme-friendly organic solvents generally have the meanings as previously set forth.

Due to the process for producing pancreatin micropellets, viz the coating process as described herein, pharmaceutically acceptable residual amounts of the enzyme-friendly organic solvent(s) present in the enteric-coating solution may still be present in the pancreatin micropellet after drying. It is understood that pancreatin micropellets comprising pharmaceutically acceptable residual amounts of enzyme-friendly organic solvent(s) are within the scope of the present invention.

Process step bb) may be performed at a temperature between about 15° C. and about 60° C. Performing process step bb) at ambient temperature (i.e. room temperature, approximately between about 20° C. and about 30° C.), is preferred. Examples of suitable enzyme-friendly organic solvents include acetone, 2-butanol, tert.-butanol, chloroform, dichloromethane, ethanol, methanol, 1-propanol, 2-propanol and mixtures of said solvents. Acetone, ethanol and 2-propanol or their mixtures are preferred as enzyme-friendly organic solvents. Acetone is most preferred. The foregoing list of enzyme-friendly organic solvents in process step bb.) is not meant to be exhaustive but merely illustrative, as a person or ordinary skill in the art would understand that many other enzyme-friendly organic solvents or combination of solvents could also be used.

The enzyme-friendly organic solvent is typically used in an amount between about 6 and about 10 times, preferably between about 7 and about 8 times, the weight of the non-solvent coating constituents used to prepare the pancreatin micropellets. For example, if the non-solvent coating constituents make up to a total weight of about 1.5 g, then about 9 g to about 15 g of enzyme-friendly organic solvent may be used in process step aa).

In process step cc.) the product temperature of the pancreatin micropellet cores, in one embodiment, is usually maintained between about 30° C. and about 60° C. while coating, preferably between about 32° C. and about 55° C., more preferred between about 35° C. and about 50° C., most preferably between about 37° C. and about 49° C. In process step cc.), when cetyl alcohol or a mixture of cetyl alcohol and triethyl citrate is used the product temperature of the pancreatin micropellet cores is maintained between about 40° C. and about 46° C. Maintaining the product temperature of the pancreatin micropellet cores within the preferred temperature ranges while coating results in improved gastric-FRDBO acid resistant properties of the pancreatin micropellets, in particular when the enteric coating comprise cetyl alcohol and triethyl citrate as plasticizers. The coating in process step cc.) can be accomplished by any process or method known to a person of ordinary skill in the art. Spray coating is preferred. Usually, process step cc.) is performed in a way that the enteric coating comprises between about 20% and about 30% by weight, preferably between about 22% and about 26% by weight and more preferably between about 22.5% and about 25% by weight of the total composition of the pancreatin micropellet. The exact parameters to be applied in process step cc.) to achieve the desired enteric coating will depend on the coating technique used. The person skilled in the art understands how to achieve coating films of a desired thickness when using different coating techniques.

Drying of the enteric-coated pancreatin micropellet cores in process step dd.) is performed between about 30° C. and about 75° C. preferably between about 30° C. and about 55° C., preferably between about 35° C. and about 50° C., and for a period of between about 6 hours and about 60 hours, preferably for a period of between about 10 hours and about 36 hours.

Pancreatin micropellets according to the invention are particularly suitable for delivery of pancreatin and its digestive enzyme constituents to the upper intestine, in particular to the small intestine, usually to the duodenum, of mammals such as humans. Thus, pancreatin micropellets according to the invention are useful for the prophylaxis and/or treatment of various medical conditions and digestive disorders including pancreatic exocrine insufficiency of different origins like maldigestion, and/or for the prophylaxis and/or treatment of pancreatitis, cystic fibrosis, diabetes type 1 and/or diabetes type II in mammals such as humans. Maldigestion in mammals such as humans is usually based on a deficiency of digestive enzymes, in particular on a deficiency of endogenous lipase, but also of protease and/or amylase. The cause of such a deficiency of digestive enzymes is frequently a hypofunction of the pancreas (e.g. pancreatic insufficiency, usually known as pancreatic exocrine insufficiency), the organ which produces the largest quantity of, and the most important, endogenous digestive enzymes. If the pancreatic insufficiency is pathological, it may be congenital or acquired. Acquired chronic pancreatic insufficiency may, for example, result from alcoholism. Congenital pancreatic insufficiency may, for example, result from disease such as cystic fibrosis. The consequences of the deficiency of digestive enzymes may be severe symptoms of under-nutrition and malnutrition, which may be accompanied by increased susceptibility to secondary illnesses. In one specific embodiment, pancreatin micropellets according to the invention are therefore particularly suited for treating pancreatic exocrine insufficiency of any origin.

In another embodiment, pancreatin micropellets are provided as previously described, for the manufacture of a medicament for the treatment of medical conditions such as digestive disorders, pancreatic exocrine insufficiency, pancreatitis, cystic fibrosis, diabetes type I and/or diabetes type II.

In yet another embodiment, a method is provided for the treatment of a medical condition such as digestive disorders, pancreatic exocrine insufficiency, pancreatitis, cystic fibrosis, diabetes type I and/or diabetes type II by administering a therapeutically effective amount of pancreatin micropellets previously described to a mammalian subject in need of such treatment.

A further embodiment includes a pharmaceutical composition comprising a pharmacologically effective amount of pancreatin wherein the pancreatin is in the form of pancreatin micropellets manufactured according to the processes described herein in a dos-age form suitable for oral administration containing said pharmacologically effective amount of pancreatin. The pancreatin micropellets described herein maybe placed into pharmaceutically acceptable dosages forms such as capsules or sachets. Capsules or sachets maybe opened to permit mixing of the contents with compatible foods or liquids to facilitate administration of the contents of the capsule or sachet.

For proper delivery of an acid-labile drug like pancreatin to the upper intestine of a mammal such as a human, it is necessary that an enteric coating be gastric acid resistant up to a pH of about 5.5. Subsequently, the acid-labile drug will need to be released to the upper intestine which means that the enteric coating must release the acid-labile drug in a less acidic environment, e.g. at about pH 5.5 or higher, in particular at a pH of about 6. The pancreatin micropellets described herein possess superior gastric acid resisting and protective properties, e.g. superior protective properties at about pH 1 and/or about pH 5. Pancreatin micropellets according to the invention wherein the plasticizer is a mixture of cetyl alcohol and triethyl citrate as described above (“CA/TEC-Compositions”) are preferred in this regard. Further, CA/TEC-Compositions in general preserve a higher lipase content and usually possess a lower water content relative to other pancreatin micropellets where other plasticizers are used. Furthermore, CA/TEC-Compositions exhibit a favorable dissolution profile which is comparable to the presently marketed pancreatin containing medicaments, e.g. to medicaments available under the trade name Creon™.

In other embodiments of the invention, a pharmaceutical pack or kit is provided comprising one or more containers filled with the pancreatin micropellets described herein. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

### Examples

The following examples are meant to be illustrative and not to limit the present disclosure. Other suitable modifications and adaptations are of the variety normally encoun-tered by those skilled in the art and are fully within the spirit and scope of the present disclosure.

### A. Preparation of Pancreatin Micropellet Cores and Pancreatin Micropellets

### 1. Preparation of Uncoated Pancreatin Micropellet Cores

15.9 kg of pancreatin was mixed with 3.975 kg of polyethylene glycol 4000 in a commercially available high shear mixer and thoroughly moistened with 3.975 kg of 2-propanol. The resulting mixture was extruded by means of a commercially available extruding press which was equipped with a piercing die having 0.8 mm internal diameter bores and a cutting device arranged downstream. The temperature was less than 50° C. while pressing. The extruded mass was cut into extrudate fragments of approximately 5 mm length by means of the cutting device.

The resulting 14.64 kg of the extrudate fragments were transferred in four portions of roughly equal size to a commercially available rounding apparatus and rounded to give approximately elliptically or approximately spherically shaped micropellet cores. An additional 135 g of 2-propanol was added while rounding.

After drying in a commercially available continuous vacuum dryer (VOtsch type) at a temperature in a range from between 35° C. and 50° C. for 12 hours, the pancreatin micropellets were graded, first with a 3.15 mm sieve (sieving of oversize grain >3.15 mm) and then with a 0.7 mm sieve (sieving of undersize grain <0.7 mm) and afterwards with a 1.25 mm sieve (sieving of oversize grain >1.25 mm) to yield 11.98 kg of pancreatin micropellet cores having a pancreatin content of 80% and a bulk density of 0.67 g/ml.

### 2. Enteric Coating of Pancreatin Micropellet Cores

A coating solution was prepared by adding 1623.2 g of hydroxypropyl methylcellulose phthalate (HP 55), 90.2 g of triethyl citrate, 34.3 g of cetyl alcohol and 38.9 g of di-methicone 1000 to 14030 g of acetone at room temperature while stirring.

5025 g of pancreatin micropellet cores (prepared analogously to the process as described herein) were fed into a commercially available fluid bed coater and were spray-coated at a spray rate of 97-101 kg/h and an air pressure of 1.7 bar with the coating solution as prepared above until the desired film-thickness of the coating had been reached. The product temperature of the pancreatin micropellet cores was monitored and maintained in the range between about 37° C. and about 43° C. during coating. The resulting pancreatin micropellets were then dried in a commercially available vacuum dryer (Vötsch type) at a temperature in a range between 35° C. and 50° C. for 12 hours. The dried pancreatin micropellets were then graded, first with a 0.7 mm sieve (sieving of undersize grain <0.7 mm) and then with a 1.6 mm sieve (sieving of oversize grain >1.6 mm) to yield 6532 g of pancreatin micropellets having a pancreatin content of about 60% relative to the enteric-coated pancreatin micropellets. The bulk density of the pancreatin micropellets was about 0.69 g/ml.

Further pancreatin micropellets were prepared according to the procedure described above and different coatings were applied in a manner similar to the coating process set forth above to yield additional pancreatin micropellets. The compositions of the additional pancreatin micropellets and certain process parameters from the coating processes are given below in Table 1. Composition G can be produced according to processes as described in U.S. Pat. No. 5,378,462. Comparative composition H was prepared according to a process as described above which has been slightly modified (i.e. dibutylphthalate was used as a plasticizer in the coating). All batches have been produced in laboratory scale except where otherwise indicated.

TABLE 1
Composition of (enteric-coated) pancreatin micropellets
and applicable process parameters

| Ingredients | | Composition | | | | | |
|---|---|---|---|---|---|---|---|
| mg/capsule | | A | B | C | D | 1 | 2 |
| Mi- | Pancre- | 150.00 | 150.00 | 150.00 | 150.00 | 150.00 | 150.00 |
| cro- | atin | | | | | | |
| pellet | PEG | 37.50 | 37.50 | 37.50 | 37.50 | 37.50 | 37.50 |
| Cores | 4000 | | | | | | |
| En- | HP 55 | 48.60 | 48.60 | 48.60 | 48.60 | 48.60 | 48.60 |
| teric | Dimeth- | 1.25 | 1.25 | 1.25 | 1.25 | 1.25 | 1.25 |
| Coat- | icone | | | | | | |
| ing | TEC | 0 | 0 | 3.0 | 4.10 | 5.00 | 0 |
| (film) | CA | 0 | 0.40 | 0 | 0 | 0 | 1.00 |
| | Sum | 237.40 | 237.75 | 240.35 | 241.45 | 242.4 | 238.35 |
| Pro- | Pellet | 40° C. | 40° C. | 40° C. | 40° C. | 40° C. | 40° C. |
| cess | temp. | | | | | | |
| para- | while | | | | | | |
| me- | coating | | | | | | |
| ters | | | | | | | |
| Ingredients | | Composition | | | | | |
| mg/capsule | | 3 | 4 | 5 | 6* | 7 | 8 |
| Mi- | Pancre- | 150.00 | 150.00 | 150.00 | 150.00 | 150.00 | 150.00 |
| cro- | atin | | | | | | |
| pellet | PEG | 37.50 | 37.50 | 37.50 | 37.50 | 37.50 | 37.50 |
| Cores | 4000 | | | | | | |
| En- | HP 55 | 52.60 | 48.60 | 48.60 | 52.25 | 52.25 | 52.25 |
| teric | Dimeth- | 1.25 | 1.25 | 1.25 | 1.25 | 1.25 | 1.25 |
| Coat- | icone | | | | | | |
| ing | TEC | 0 | 3.60 | 3.00 | 2.90 | 2.90 | 2.90 |
| (film) | CA | 1.15 | 0.40 | 1.00 | 1.10 | 1.10 | 1.10 |
| | Sum | 242.50 | 241.35 | 241.35 | 245.00 | 245.00 | 245.00 |
| Pro- | Pellet | 40° C. | 40° C. | 40° C. | 40° C. | 30° C. | 35° C. |
| cess | temp. | | | | | | |
| para- | while | | | | | | |
| me- | coating | | | | | | |
| ters | | | | | | | |
| Ingredients | | Composition | | | | | |
| mg/capsule | | 9 | 10 | 11 | 12 | 13 | 14 |
| Mi- | Pancre- | 150.00 | 150.00 | 150.00 | 150.00 | 150.00 | 150.00 |
| cro- | atin | | | | | | |
| pellet | PEG | 37.50 | 37.50 | 37.50 | 37.50 | 37.50 | 37.50 |
| Cores | 4000 | | | | | | |
| En- | HP 55 | 56.34 | 56.34 | 56.34 | 52.25 | 52.25 | 56.34 |
| teric | Dimeth- | 1.35 | 1.35 | 1.35 | 1.25 | 1.25 | 1.35 |
| Coat- | icone | | | | | | |
| ing | TEC | 3.13 | 3.13 | 3.13 | 2.90 | 2.90 | 3.13 |
| (film) | CA | 1.19 | 1.19 | 1.19 | 1.10 | 1.10 | 1.19 |
| | Sum | 249.51 | 249.51 | 249.51 | 245.00 | 245.00 | 249.51 |
| Pro- | Pellet | 37° C. | 40° C. | 43° C. | 49° C. | 40° C. | 46° C. |
| cess | temp. | | | | | | |
| para- | while | | | | | | |
| me- | coating | | | | | | |
| ters | | | | | | | |

| | | Composition | | | | |
|---|---|---|---|---|---|---|
| Ingredients mg/capsule | | 15 | E | F | G | H |
| Micropellet | Pancreatin | 128.06 | 150.00 | 150.00 | 150.00 | 150.00 |
| | PEG 4000 | 32.01 | 37.50 | 37.50 | 37.50 | 37.50 |
| Cores | Light | 0 | 0 | 0 | 3.75 | 0 |
| | mineral | | | | | |
| | oil | | | | | |
| Enteric | HP 55 | 48.10 | 48.60 | 48.60 | 48.60 | 48.60 |
| Coating | Dimethi- | 1.15 | 1.25 | 1.25 | 1.25 | 1.25 |
| (film) | cone | | | | | |
| | TEC | 2.67 | 1.00 | 2.00 | 0 | 0 |
| | CA | 1.01 | 0 | 0 | 0 | 0 |
| | DBP | 0 | 0 | 0 | 4.10 | 4.10 |
| | Light | 0 | 0 | 0 | 3.30 | 0 |
| | mineral oil | | | | | |
| | Sum | 213.00 | 238.35 | 239.35 | 248.50 | 241.50 |
| Process | Pellet | n.a. | 40° C. | 40° C. | 40° C. | 40° C. |
| parameters | temp. | | | | | |
| | while | | | | | |
| | coating | | | | | |

PEG = polyethylene glycol;
TEC = triethyl citrate;
CA = cetyl alcohol;
HP 55 = hydroxypropyl methylcellulose phthalate;
temp. = temperature;
DBP = dibutyl phthalate;
* = production scale; n.a.: data not available.

Composition G is a currently available high-quality pharmaceutical composition comprising pancreatin and light mineral oil.

Compositions No. 6, 10, 13, 14 and 15 are examples of CA/TEC compositions.

Composition No. 3 is an example of a composition comprising cetyl alcohol as the plasticizer.

### B. Determination of the Gastric Acid Resistance of Enteric-Coated Pancreatin Micropellets at pH 1 and pH 5

The gastric acid resistances of the pancreatin micropellet s (see Table 1 hereabove) were measured.

Resistance to gastric juice (pH 1) of the different pancreatin micropellets from Table 1 was determined by immersing the pancrelipase micropellets for 2 hours in 0.1 mol/1 hy-drochloric acid in a disintegration tester according to the European Pharmacopoeia (Ph. Eur.). Then the un-dissolved portion of the pellets was separated from the solution and their residual lipase activity was determined according to the lipase assay of Ph. Eur./The International Pharmaceutical Federation” (FIP), PO Box 84200; 2508 AE The Hague; The Netherlands. The results of these tests for gastric resistance of the enteric coating are presented in Table 2 (“stability at pH 1”).

Further, a similar test at pH 5 was performed using the same conditions as outlined above, with the exception that a phosphate buffer pH 5.0 (2.0 g sodium chloride and 9.2 g sodium di-hydrogen phosphate monohydrate per liter adjusted to pH 5.0) was used as a solvent instead of 0.1 mol/I hydrochloric acid. The results of these tests for gastric resistance are also presented below in Table 2 (“stability at pH 5”).

The gastric acid resistances of the compositions from Table 1 (see above) are each given in Table 2 as percentages of the residual lipolytic activity after the incubation in rela-tion to the actual lipolytic activity of the samples tested prior to the incubation (relative gastric acid resistance). The lipolytic activity is determined according to the lipase assay described in the USP monograph “pancrelipase delayed-release capsules”. In principle, any standardized and characterized pancreatin sample may be used as the lipase reference standard. For example, a predetermined lipolytic activity standard may be obtained from the “International Pharmaceutical Federation” (FIP), PO Box 84200; 2508 AE The Hague; The Netherlands. For the purposes of the present invention, an internal pancreatin standard was used which is available on request from Solvay Pharmaceuticals GmbH, Hans-Boeckler-Allee 20, 30173 Hannover, Germany.

TABLE 2
Relative gastric acid resistances (stabilities)
of the pancreatin micropellets at pH 1 and pH 5

| | Stability | Stability |
|---|---|---|
| | at pH 5 | at pH 1 |
| Composition | [%] | [%] |
| A | 15.3 | 15.9 |
| B | 63.2 | 53.8 |
| C | 71.6 | 84.2 |
| D | 52.0 | 93.6 |
| 1 | 87.0 | 96.0 |
| 2 | 76.4 | 92.6 |
| 3 | 92.1 | 94.5 |
| 4 | 85.3 | 93.7 |
| 5 | 92.0 | 93.0 |
| 6 | 94.9 | 99.4 |
| 7 | 67.4 | 89.8 |
| 8 | 80.5 | 95.2 |
| 9 | 83.8 | 90.8 |
| 10 | 97.9 | 99.6 |
| 11 | 89.0 | 93.5 |
| 12 | 83.7 | 94.8 |
| 13 | 100.2 | 102.7 |
| 14 | 93.6 | 98.7 |
| E | 48.6 | 65.0 |
| F | 36.5 | 75.0 |
| G | 98.6 | 100.6 |

Preferred pancreatin micropellets have a gastric acid resistance (stability) at pH 1 of at least 75%, in particular of at least 85%, preferably of at least 90%, more preferred of at least 95%, relative to a predetermined pancreatin lipolytic activity standard.

Other preferred pancreatin micropellets as disclosed herein have a gastric acid resistance at pH 5 of at least 75%, in particular of at least 85%, preferably of at least 90%, more preferred of at least 95%, relative to a predetermined pancreatin lipolytic activity standard.

Pancreatin micropellets which are most preferred have a gastric acid resistance at pH 1 of at least 90% and an additional gastric acid resistance at pH 5 of at least 90%, relative to a predetermined pancreatin lipolytic activity standard.

### C. Determination of the Dissolution Profile of Enteric Coated Pancreatin Micropellets

The dissolution profile of different enteric coated pancreatin micropellets from Table 1 (see above) was determined according to a test procedure as described in the United States Pharmacopoeia (USP) monograph “pancrelipase delayed-release capsules” with increased gastric resistance phase which is hereby incorporated by reference.

The determination of the resistance to gastric fluid was performed using gastric juice without enzymes according to USP under standardized conditions (37° C., 100 rpm) for 2 hours in the dissolution apparatus (basket apparatus USP). Then the un-dissolved portion of the enteric coated pancreatin micropellets was separated from the solution and transferred into the paddle apparatus according to USP, filled with phosphate buffer solution at pH 6.0 to determine the dissolution of enzymes. The enteric coated pancreatin micropellets were agitated in a dissolution tester under standardized conditions for usually 90 minutes (see exact timepoints in Table 3 below) at 37° C. and 50 rpm.

The lipase activity was determined after selected time points (see Table 3) according to the lipase assay described in the USP monograph “pancrelipase delayed-release capsules”.

The results of the dissolution profile test are presented as “% residual lipase activity of actual lipase activity” below (see Table 3).

TABLE 3
Dissolution profiles of the enteric coated
pancreatin micropellets in phosphate buffer

| Time | % lipase activity of initial actual activity | | |
|---|---|---|---|
| points | for pancreatin micropellet composition No. | | |
| [min.] | G | H | 14 |
| 5 | 0.0 | 4 | NA |
| 10 | 0.0 | 6.25 | 15.37 |
| 15 | 11.9 | 23.15 | 34.38 |
| 20 | 48.0 | 48.15 | NA |
| 25 | 62.3 | 62.9 | NA |
| 30 | 73.5 | 69.6 | 73.86 |
| 45 | 77.1 | 77.15 | 84.45 |
| 60 | 79.9 | 78.35 | 81.25 |
| 75 | 78.4 | 76.7 | 80.40 |
| 90 | 78.2 | 75.25 | NA |

For the dissolution profile test results as provided in Table 3, a comparison of the compositions G, H and 14 was performed. The comparison was based on the “Guidance for Industry”, SUPAC-MR, Modified Release Solid Oral Dosage Forms (September 1997) by calculating the similarity factor (f2). The 2 acceptance limits for determining similarity of two compared curves were (i) a factor (f2)>50 and (ii) the average deviation at any dissolution sampling point should not be greater than 15%.

In vitro dissolution profile comparisons can be made using a model independent approach using similarity factor. Dissolution profiles may be compared using the following equation that defines a similarity factor (f2):

f₂=50 log{[1+1/nΣⁿₜ₌₁(Rₜ−Tₜ)²]^{-0.5}*100}
where log=logarithm to base 10, n=number of sampling time points, Σ=summation over all time points, Rₜ=dissolution at time point t of the reference (unchanged drug product, i.e. prechange batch), Tₜ=dissolution at time point t of the test (changed drug product, i.e., post-change batch).

When applying the above-stated acceptance limits for determining similarity (f2=71.8) it was found that the dissolution profile of pancreatin micropellet composition “H” (see Table 1) could be considered to be similar to the dissolution profile of the reference pancreatin micropellet composition “G” (see Table 1).

All references, including publications, patent applications, and patents, cited herein are hereby incorporated by reference to the same extent as if each reference there individually and specifically indicated to be incorporated by reference were set forth in its entirety herein.

The use of the terms “a” and “an” and “the” and similar references in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the invention.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

The use of individual numerical values are stated as approximations as though the values were preceded by the word “about” or “approximately” unless clearly indicated otherwise by context. Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the claimed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately”. Thus, recitation of ranges of values herein are merely intended to serve as a shorthand method of referring individually to each separate value falling within the range, unless otherwise indicated herein, and each separate value is incorporated into the specification as if it were individually recited herein.

We claim:

1. A process for the manufacture of pancreatin micropellet cores, comprising the steps of:
a. preparing an extrudable mixture comprising:
i. about 10% to about 95% pancreatin;
ii. about 5% to about 90% of at least one pharmaceutically acceptable binding agent;
iii. 0% to about 10% of at least one pharmaceutically acceptable excipient; and
iv. one or more enzyme-friendly organic solvents in an amount sufficient to form an extrudable mixture;
wherein the percentages of components are weight to weight of the pancreatin micropellet cores;
b. creating pancreatin micropellet cores from the extrudable mixture;
c. forming the pancreatin micropellet cores into approximately spherical or approximately ellipsoidal shape in the presence of additional enzyme-friendly organic solvent; and
d. removing the one or more enzyme-friendly organic solvents from the pancreatin micropellet cores such that the pancreatin micropellet cores are substantially free of the one or more enzyme-friendly organic solvents;
wherein the pancreatin micropellet cores are substantially free of synthetic oils.

2. The process of claim 1 wherein the pancreatin is present between about 70% and about 90% weight to weight of the pancreatin micropellet cores.

3. The process of claim 1 wherein the binding agent is present between about 10% and about 30% weight to weight of the pancreatin micropellet cores.

4. The process of claim 1 wherein the pharmaceutically acceptable excipient is present between about 0% and about 5% weight to weight of the pancreatin micropellet cores.

5. The process of claim 1 wherein the binding agent is selected from the group consisting of: polyethylene glycol 1500, polyethylene glycol 2000, polyethylene glycol 3000, polyethylene glycol 4000, polyethylene glycol 6000, polyethylene glycol 8000, polyethylene glycol 10000, hydroxypropyl methylcellulose, polyoxyethylen, copolymers of polyoxyethylen-polyoxypropylen and mixtures of said organic polymers.

6. The process of claim 1 wherein the binding agent is polyethylene glycol 4000.

7. The process of claim 1 wherein the at least one pharmaceutically acceptable excipient is selected from the group consisting of: magnesium stearate, calcium stearate, stearic acid, talcum, starch, calcium phosphate, corn starch, dextrans, dextrin, hydrated silicon dioxide, microcrystalline cellulose, kaolin, lactose, mannitol, polyvinyl pyrrolidone, precipitated calcium carbonate, sorbitol, silicic acid, alginic acid, amylose, calcium alginate, calcium carbonate, formaldehyde gelatin, pectic carbonate, sago starch, sodium bicarbonate and glycerol.

8. The process of claim 1 wherein the one or more enzyme-friendly organic solvents are present between about 15% and about 35% by weight relative to the amount of pancreatin.

9. The process of claim 1 wherein the one or more enzyme-friendly organic solvents are present in an amount of less then about 5% weight to weight of the pancreatin micropellet cores after the one or more enzyme-friendly organic solvents have been removed from the pancreatin micropellet cores.

10. The process of claim 1 wherein the one or more enzyme-friendly organic solvents is selected from the group consisting of: acetone, chloroform, dichloromethane, methanol, ethanol, 1-propanol, 2-propanol, 2-butanol, tert-butanol and mixtures of said solvents.

11. The process of claim 1 wherein the one or more enzyme-friendly organic solvents is 2-propanol.

12. The process of claim 1 wherein removing of the one or more enzyme-friendly organic solvents from the pancreatin micropellet cores is by drying at a temperature between about 30° C. and about 75° C.

13. The process of claim 1 wherein creating the pancreatin micropellet cores is by extrusion.

14. The process of claim 1 wherein forming of the pancreatin micropellet cores is performed in a rounding apparatus.

15. A process for the manufacture of pancreatin micropellets, comprising the steps of:
aa. providing pancreatin micropellet cores wherein the pancreatin micropellet cores are substantially free of synthetic oils;
bb. providing an enteric-coating solution comprising
i. one or more film-forming agents;
ii. a plasticizer in an amount greater than about 1.5% by weight relative to the one or more film-forming agents film-forming agents; and
iii. optionally, at least one anti-sticking agent, and
iv. one or more enzyme-friendly organic solvent(s);
cc. coating the pancreatin micropellet cores with the enteric-coating solution wherein the temperature of the pancreatin micropellet cores during coating is kept at a temperature suitable for applying the enteric-coating solution; and
dd. drying the coated pancreatin micropellet cores.

16. The process of claim 15 wherein the enteric coating is between about 20% and about 30% by weight of the pancreatin micropellets.

17. The process of claim 15 wherein the one or more film-forming agents is selected from the group consisting of: agar, carbomer polymers, carboxymethyl cellulose, carboxymethylethyl cellulose, carrageen, cellulose acetate phthalate, cellulose acetate succinate, cellulose acetate trimelliate, chitin, corn protein extract, ethyl cellulose, gum arabic, hydroxypropyl cellulose, hydroxypropylmethyl acetate succinate, hydroxypropyl methylcellulose acetate succinate, hydroxypropyl methylcellulose phthalate, methacrylic acid-ethyl methacrylate-copolymer, methyl cellulose, pectin, polyvinyl acetate phthalate, polivinyl alcohol, shellac, sodium alginate, starch acetate phthalate, styrene/maleic acid copolymer and mixtures of said film-forming polymers.

18. The process of claim 15 wherein the plasticizer is selected from the group consisting of: saturated linear monohydric alcohols having 12 to 30 carbon atoms, lauryl alcohol, tridecyl alcohol, myristyl alcohol, pentadecyl alcohol, cetyl alcohol, heptadecyl alcohol, stearyl alcohol, nonadecyl alcohol, arachic alcohol, behenyl alcohol, carnaubyl alcohol, ceryl alcohol, corianyl alcohol, melissyl alcohol, acetyl tributyl citrate, dibutyl sebacate, fatty acid esters of glycerol, glycerol, polyethylene glycol, propyleneglycol, sorbitan fatty acids, triacetin, triethyl citrate and mixtures of any of said plasticizers.

19. The process of claim 15 wherein the plasticizer is cetyl alcohol.

20. The process of claim 15 wherein the plasticizer is triethyl citrate present in an amount of between about 5% and about 20% by weight relative to the film-forming agent.

21. The process of claim 15 wherein the plasticizer is comprised of cetyl alcohol and triethyl citrate which are collectively present in an amount greater than about 3% by weight relative to the film-forming agent.

22. The process of claim 15 wherein the plasticizer is comprised of cetyl alcohol and triethyl citrate which are collectively present in an amount between about 4% and about 20% by weight relative to the film-forming agent.

23. The process of claim 22 wherein the ratio of cetyl alcohol to triethyl citrate is between about 0.05:1 and about 1:1 by weight.

24. The process of claim 15 wherein the anti-sticking agent is selected from the group consisting of: dimethicone and castor oil.

25. The process of claim 15 wherein the anti-sticking agent is present in an amount between about 1.5% and about 3% by weight relative to the film-forming agent.

26. The process of claim 15 wherein the one or more enzyme-friendly organic solvents is selected from the group consisting of: acetone, chloroform, dichloromethane, methanol, ethanol, 1-propanol, 2-propanol, 2-butanol, tert-butanol and mixtures of said solvents.

27. A method of treating a medical condition in a mammalian subject, comprising the steps of:
a. providing pancreatin micropellet cores manufactured according to the process of claim 1 in a dosage form suitable for oral administration; and
b. orally administering the dosage form to the subject to provide pancreatin in an amount sufficient to treat the medical condition;
wherein the medical condition is selected from the group consisting of: pancreatic exocrine insufficiency, pancreatitis, cystic fibrosis, diabetes type I and diabetes type II.

28. A method of treating a medical condition in a mammalian subject, comprising the steps of:
a. providing pancreatin micropellets manufactured according to the process of claim 15 in a dosage form suitable for oral administration; and
b. orally administering the dosage form to the subject to provide pancreatin in an amount sufficient to treat the medical condition;
wherein the medical condition is selected from the group consisting of: pancreatic exocrine insufficiency, pancreatitis, cystic fibrosis, diabetes type I and diabetes type II.

29. A pharmaceutical composition, comprising
a. a pharmacologically effective amount of pancreatin wherein said pancreatin is in the form of pancreatin micropellet cores manufactured according to the process of claim 1; and
b. a dosage form suitable for oral administration containing said pharmacologically effective amount of pancreatin.

30. A pharmaceutical composition, comprising
a. a pharmacologically effective amount of pancreatin wherein said pancreatin is in the form of pancreatin micropellets manufactured according to the process of claim 15; and
b. a dosage form suitable for oral administration containing said pharmacologically effective amount of pancreatin.

31. A pharmaceutical composition, prepared by a process comprising the steps of:
a. preparing an extrudable mixture comprising:
i. about 10% to about 95% pancreatin;
ii. about 5% to about 90% of at least one pharmaceutically acceptable binding agent;
iii. 0% to about 10% of at least one pharmaceutically acceptable excipient; and
iv. one or more enzyme-friendly organic solvents in an amount sufficient to form an extrudable mixture;
wherein the percentages of components are weight to weight of the pancreatin micropellet cores;
b. creating pancreatin micropellet cores from the extrudable mixture;
c. forming the pancreatin micropellet cores into approximately spherical or approximately ellipsoidal shape in the presence of additional enzyme-friendly organic solvent;
d. removing the one or more enzyme-friendly organic solvents from the pancreatin micropellet cores such that the pancreatin micropellet cores are substantially free of the one or more enzyme-friendly organic solvents;
wherein the pancreatin micropellet cores are substantially free of synthetic oils;
e. coating the pancreatin micropellet cores with an enteric-coating solution wherein the temperature of the pancreatin micropellet cores during coating is is kept at a temperature suitable to apply the enteric-coating solution;
f. drying the coated pancreatin micropellet cores; and
g. placing the coated pancreatin micropellet cores in a dosage form suitable for oral administration.

32. The process of claim 31 wherein the pancreatin is present between about 70% and about 90% weight to weight of the pancreatin micropellet cores.

33. The process of claim 31 wherein the binding agent is present between about 10% and about 30% weight to weight of the pancreatin micropellet cores.

34. The process of claim 31 wherein the binding agent is selected from the group consisting of: polyethylene glycol 1500, polyethylene glycol 2000, polyethylene glycol 3000, polyethylene glycol 4000, polyethylene glycol 6000, polyethylene glycol 8000, polyethylene glycol 10000, hydroxypropyl methylcellulose, polyoxyethylen, copolymers of polyoxyethylen-polyoxypropylen and mixtures of said organic polymers.

35. The process of claim 31 wherein the binding agent is polyethylene glycol 4000.

36. The process of claim 31 wherein the at least one pharmaceutically acceptable excipient is selected from the group consisting of: magnesium stearate, calcium stearate, stearic acid, talcum, starch, calcium phosphate, corn starch, dextrans, dextrin, hydrated silicon dioxide, microcrystalline cellulose, kaolin, lactose, mannitol, polyvinyl pyrrolidone, precipitated calcium carbonate, sorbitol, silicic acid, alginic acid, amylose, calcium alginate, calcium carbonate, formaldehyde gelatin, pectic carbonate, sago starch, sodium bicarbonate and glycerol.

37. The process of claim 31 wherein the one or more enzyme-friendly organic solvents are present between about 15% and about 35% by weight relative to the amount of pancreatin.

38. The process of claim 31 wherein the one or more enzyme-friendly organic solvents is selected from the group consisting of: acetone, chloroform, dichloromethane, methanol, ethanol, 1-propanol, 2-propanol, 2-butanol, tert-butanol and mixtures of said solvents.

39. The process of claim 31 wherein the one or more enzyme-friendly organic solvents is 2-propanol.

40. The process of claim 31 wherein removing the one or more enzyme-friendly organic solvents from the pancreatin micropellet cores is by drying at a temperature between about 30° C. and about 75° C.

41. A pharmaceutical composition, prepared by a process comprising the steps of:
a. providing pancreatin micropellet cores wherein the pancreatin micropellet cores are substantially free of synthetic oils;
b. providing an enteric-coating solution comprising
i. at least one film-forming agent
ii. a plasticizer in an amount of greater than about 1.5% by weight relative to the one or more film-forming agents film-forming agents; and
iii. optionally at least one anti-sticking agent in one or more enzyme-friendly organic solvent;
c. coating the pancreatin micropellet cores with the enteric-coating solution wherein the temperature of the pancreatin micropellet cores during coating is kept at a temperature suitable to apply the enteric-coating solution; and
d. drying the coated pancreatin micropellet cores; and
e. placing the coated pancreatin micropellet cores in a dosage form suitable for oral administration.

42. The composition of claim 41 wherein the enteric coating is between about 20% and about 30% by weight of the pancreatin micropellets.

43. The composition of claim 41 wherein the one or more film-forming agents is selected from the group consisting of: agar, carbomer polymers, carboxymethyl cellulose, carboxymethylethyl cellulose, carrageen, cellulose acetate phthalate, cellulose acetate succinate, cellulose acetate trimelliate, chitin, corn protein extract, ethyl cellulose, gum arabic, hydroxypropyl cellulose, hydroxypropylmethyl acetate succinate, hydroxypropyl methylcellulose acetate succinate, hydroxypropyl methylcellulose phthalate, methacrylic acid-ethyl methacrylate-copolymer, methyl cellulose, pectin, polyvinyl acetate phthalate, polivinyl alcohol, shellac, sodium alginate, starch acetate phthalate, styrene/maleic acid copolymer and mixtures of said film-forming polymers.

44. The composition of claim 41 wherein the plasticizer is selected from the group consisting of: saturated linear monohydric alcohols having 12 to 30 carbon atoms, lauryl alcohol, tridecyl alcohol, myristyl alcohol, pentadecyl alcohol, cetyl alcohol, heptadecyl alcohol, stearyl alcohol, nonadecyl alcohol, arachic alcohol, behenyl alcohol, carnaubyl alcohol, ceryl alcohol, corianyl alcohol, melissyl alcohol, acetyl tributyl citrate, dibutyl sebacate, fatty acid esters of glycerol, glycerol, polyethylene glycol, propyleneglycol, sorbitan fatty acids, triacetin, triethyl citrate and mixtures of any of said plasticizers.

45. The composition of claim 41 wherein the plasticizer is cetyl alcohol.

46. The composition of claim 41 wherein the plasticizer is triethyl citrate present in an amount of between about 5% and about 20% by weight relative to the film-forming agent.

47. The composition of claim 41 wherein the plasticizer is a mixture of cetyl alcohol and triethyl citrate which are collectively present in an amount of greater than about 3% by weight relative to the film-forming agent.

48. The composition of claim 47 wherein the ratio of cetyl alcohol to triethyl citrate is between about 0.05:1 and about 1:1 by weight.

49. The composition of claim 41 wherein the anti-sticking agent is selected from the group consisting of: dimethicone and castor oil.

50. The composition of claim 41 wherein the anti-sticking agent is present in an amount between about 1.5% and about 3% by weight relative to the film-forming agent.

51. The composition of claim 41 wherein the one or more enzyme-friendly organic solvents is selected from the group consisting of: acetone, chloroform, dichloromethane, methanol, ethanol, 1-propanol, 2-propanol, 2-butanol, tert-butanol and mixtures of said solvents.

52. A pharmaceutical composition comprising:
pancreatin micropellets wherein the pancreatin micropellets are substantially free of synthetic oils and wherein said pancreatin micropellets have a gastric acid resistance of about 75% or more at about a pH 1.

53. A pharmaceutical composition comprising:
pancreatin micropellets wherein the pancreatin micropellets are substantially free of synthetic oils and wherein said pancreatin micropellets have a gastric acid resistance of about 75% or more at about a pH 5.

54. A pharmaceutical composition comprising:
a. pancreatin micropellet cores wherein the pancreatin micropellet cores are substantially free of synthetic oils;
b. at least one film-forming agent;
c. a plasticizer in an amount greater than about 1.5% by weight relative to the one or more film-forming agents; and
d. optionally at least one anti-sticking agent in one or more enzyme-friendly organic solvent.
